(19) United States
(12) Patent Application Publication
(10) Pub. No.: US 2005/0084492 A1
(43) Pub. Date: Apr. 21, 2005

(54) Assay method to monitor the response of anti-cytokine therapy

(76) Inventors: Mohamed Zaki, Audubon, PA (US); Suzanne Mount, Ridley Park, PA (US); Mohit Trikha, Paoli, PA (US); Jeffrey Nemeth, Havertown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/909,499

(22) Filed: Aug. 2, 2004

Related U.S. Application Data
(60) Provisional application No. 60/491,701, filed on Aug. 1, 2003.

(51) Int. Cl. G01N 33/53; A61K 39/395; G01N 33/567

(52) U.S. Cl. 424/145.1; 435/7.2

(57) ABSTRACT

A method of monitoring the response to anti-cytokine therapy in a patient undergoing such therapy by using Serum Amyloid A (SAA) protein as a biomarker.

### CROSS REFERENCE TO RELATED APPLICATION

This application is a non-provisional application filed under 37 CFR 1.53(b)(1), claiming priority under 35 USC 119(e) to provisional application No.60/491,701 filed Aug. 01, 2003, the contents of which are incorporated herein by reference.

### BACKGROUND OF THE INVENTION

1. Field of the Invention

The invention relates to an assay method for monitoring the biological response to anti-cytokine therapy. Specifically, the invention relates to a method of monitoring the response to anti-cytokine therapy using Serum Amyloid A as a biomarker.

2. Background and Related Art

There is a growing need to develop a sensitive and reliable biomarker to detect the response to anti-cytokine therapies. Currently, the only available method to detect the bioactivity of anti-cytokine therapy is measuring the levels of the target cytokine in the host serum. This approach might be the most direct. However, it can be misleading and inaccurate. In some cases the anti-cytokine drugs and the target cytokine can form immunocomplexes causing accumulation of the neutralized target in the serum of the host (Vann Zanen et al, Br J Haematol. 1998 August; 102(3):783-90). This phenomenon will lead to false detection of higher levels of target cytokine in host serum suggesting that finding an accurate and reliable biomarker for anti-cytokine based therapies is an eminent need.

The mammalian acute phase response is the first line of systemic defense elicited by stimuli such as infection, trauma, myocardial infarction, neoplasms, and surgery. It is initiated and maintained by a large number of pro-inflammatory mediators including cytokines, glucocorticosteroids and anaphylatoxins and involves a wide range of complex physiological changes including elevated circulating concentrations of hepatically synthesized acute phase reactants (APRs). In man, this latter class includes the “major” APRs, serum amyloid A (SAA) and C-reactive protein (CRP) (reviewed by Steel, D. M. and Whitehead, A. S. (1994) Immunol. Today (England) 15, 81). Serum amyloid A (SAA) is a major acute-phase protein that is produced mainly by the liver during trauma, infections, inflammation and neoplasia (Urieli-Shoval et al, Curr Opin Hematol. 2000 January; 7(1):64-9).

Several cytokines have been described in association with SAA in different disease status. IL-6 was shown to induce acute phase proteins including CRP and SAA in patients and preclinical models. SAA and IL-6 levels from renal cell cancer (RCC) patients are significantly higher than healthy donors and SAA levels correlate with poor prognosis (Kimura et al Cancer. 2001 Oct. 15; 92(8):2072-5). Other cytokines such as TNFα, IL-1, IL-2, IFNγ, ciliary neurotrophic factor, IL-11, LIF, oncostatin M and cardiotrophin were shown to induce SAA both in human and in murine models (Uhlar et al, Eur. J. Biochem. 265, 501-523, 1999). Expression of SAA was demonstrated in several disease status, including inflammatory, infectious and neoplastic diseases (Urieli-Shoval et al, Curr Opin Hematol. 2000 January; 7(1):64-9). Also, SAA mRNA was detected in several malignant cell lines such as HepG2, a hepatoma cell line, TH-1 a monocytic leukemia cell line, Sw620, a metastatic colon tumor cell line.

### SUMMARY OF THE INVENTION

The invention relates to a method to monitor the response to anti-cytokine therapy by measurement of SAA as a cytokine-responsive marker in the serum or in the diseased tissue.

Because of the association between SAA and several cytokines, SAA can be used as a direct biomarker to monitor response to anti-cytokine therapies. We have discovered that tumor cells can be induced to produce SAA, and anti-cytokine therapy such as anti-IL-6 antibody therapy can inhibit tumor cell secreted SAA. This is novel as previously it was believed that anti-cytokine therapy can decrease liver produced active protein but not tumor-produced acute phase protein such as SAA. This method is more accurate and specific than the current available method for detecting bioactivity of anti-cytokine therapies.

Thus, in accordance with the invention, there is provided a method of monitoring the response to anti-cytokine therapy in a patient undergoing such therapy by using Serum Amyloid A (SAA) protein as a biomarker, which comprises:
(a) Determining the level of SAA protein in the serum or diseased tissue of a patient;
(b) Administering the anti-cytokine therapy to the patient;
(c) Measuring the level of SAA protein in the serum or diseased tissue of the patient and determining whether the anti-cytokine therapy is effective in reducing the level of SAA protein.

### DETAILED DESCRIPTION

The present invention thus provides a new approach to monitoring anti-cytokine therapy by detecting production of SAA protein by diseased tissue, for example, tumor-cell produced SAA, and by monitoring levels of SAA, one can evaluate anti-cytokine bioactivity.

Correlations between SAA and disease progression have been described in literature. Expression of SAA was demonstrated in several disease states, including inflammatory, infectious and euplastic diseases (Urieli-Shoval et al, Curr Opin Hematol. 2000 January; 7(1):64-9). Also, SAA mRNA was detected in several malignant cell lines such as HepG2, a hepatoma cell line, TH-1 a monocytic leukemia cell line, Sw620, a metastatic colon tumor cell line. Thus, the invention may be employed in the therapy of any of these disease states where anti-cytokine therapy is employed. Various cytokines such as IL-6, TNFα, IL-1, IL-2, IFNγ, ciliary neurotrophic factor, IL-11, LIF, oncostatin M and cardiotrophin were shown to induce SAA both in human and in murine models (Uhlar et al, Eur. J. Biochem. 265, 501-523, 1999). Accordingly, the method can be used to monitor any therapy designed to neutralize or inhibit the effect of such cytokines in the afore-mentioned disease states.

In one embodiment, the improvement is to use levels of SAA as a tumor-responsive marker for anti-cytokine therapy.

Measurements of cytokine levels do not provide an accurate picture of cytokine activity. This problem is further complicated by anti-cytokine therapy, where circulating levels of cytokine/antibody complexes can be elevated. Measurement of the cytokine-responsive marker SAA in the tumor and in the serum will provide feedback as to the neutralization of the target cytokine. Levels of SAA will provide information about the response of the diseased tissues to anti-cytokine therapy.

Several methods have been reported for the measurement of SAA levels and any of these methods may be employed in the invention. These include (i) radioimmunoassays and single radial immunodiffusion procedures (Chambers, R. E. and Whicher, J. T. (1983); J. Immunol. Methods 59, 95; Marhaug, G. (1983) supra; Taktak, Y. S. and Lee, M. A. (1991); J. Immunol. Methods 136, 11); (ii) ELISA based assays (Zuckerman, S. H. and Suprenant, Y. M.(1986); J. Immunol. Methods 92, 3743; Dubois, D. Y. and Malmendier, C. L. (1988); J. Immunol. Methods 112, 71-75; Sipe, J. D. et al. (1989); J. Immunol. Methods 125, 125-135; Yamada, T. et al. (1989); Clin. Chim. Acta 179, 169-176; Tino-Casl, M. and Grubb, A. (1993); Arm. Clin. Biochem 30, 278-286); (iii) nephelometric methods (Vermeer, H. et al. (1990); Clin. Chem 36, 1192; Yamada, T. et al. (1993); Ann. Clin. Biochem. 30, 72-76); (iv) an electrophoretic procedure (Godenir, N. L. et al. (1985); J. Immunol. Methods 83, 217); (v) an immunochemiluminescence procedure (Hachem, H. et al. (1991); Clin. Biochem 24, 143-147); (vi) an automated method based on a monoclonal-polyclonal antibody solid phase enzymeimmunoassay (Wilkins, J. W. et al. (1994); Clin. Chem 40(7), 1284-1290); and (vii) time-resolved fluorometric immunoassay (Malle, E., et al. (1995); J. Immunol. Methods 182, 131). See U.S. Pat. No. 6,194,163 which discloses a method for the quantitative measurement of human acute phase serum amyloid A protein.

The following experiments demonstrate that SAA is secreted from diseased tissues and that SAA is a tumor-produced and accurate biomarker to monitor the response to anti-cytokine therapies such as anti-IL-6 antibody therapy.

### EXAMPLE 1

### Detection of Serum Amyloid A (SAA) by immunostaining on Paraffin Sections

Paraffin tumor tissue arrays were purchased from lmgenex. Slides were deparaffinized in xylene and rehydrated through graded alcohols. Slides were blocked in hydrogen peroxide (1:10 in MeOH) for 3 min. After rinsing in PBS, all slides were blocked 30 min with normal horse serum according to instructions included in the Vectastain kit (Vector, Burlingame, Calif.). Sections were incubated with anti-SAA monoclonal antibody (clone REU-86.2, Research Diagnostics, Inc.; 1:10 dilution in blocking solution), 60 min at RT. After washing in PBS, sections were incubated with biotinylated anti-mouse reagent, followed by ABC reagent, all from Vectastain Elite kit. Immunoreactivity was detected by a color reaction using DAB (Vector). Slides were counterstained with hematoxylin and mounted.

SAA could be detected in tumor tissues by immunostaining:

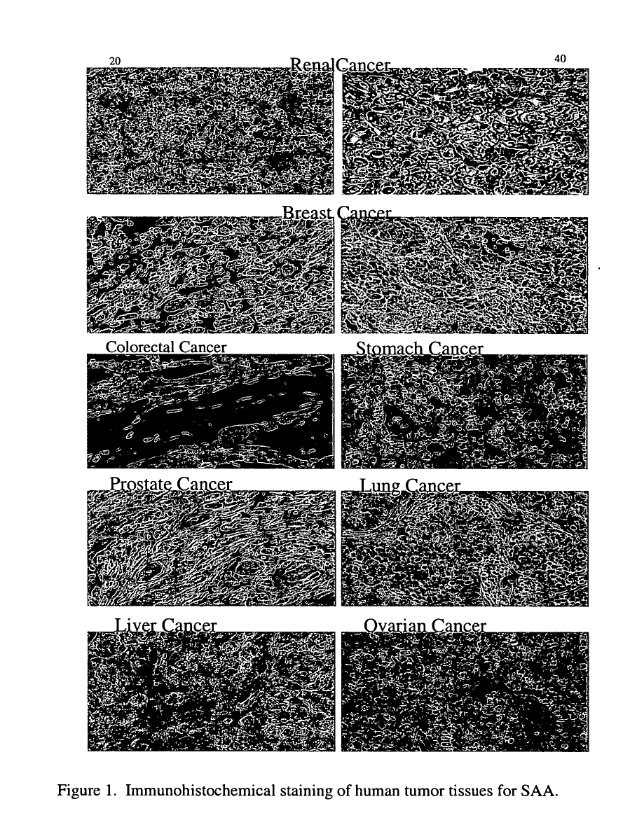

Using immunohistochemical analysis we found intense staining for SAA in many tumor tissues. Both cytoplasmic and membrane staining were observed. Specimens of renal cell carcinoma, prostate carcinoma, breast carcinoma, colorectal carcinoma, hepatic carcinoma, and squamous-type carcinoma of skin and esophagus stained intensely. Little or no staining was seen in stomach cancer, cervical cancer, ovarian cancer or lung cancer (FIG. 1).

### EXAMPLE 2

### Induction of SAA Production

A-498, a renal carcinoma cell line was purchased from ATCC (Rockville, Md.). Cells were maintained according to ATCC instructions in log phase of cell growth in DMEM/10% heat-inactivated FBS, 1% L-Gut, 1% NEAA and 1% NaPyr. CNTO 328, an anti-human IL-6 antibody developed at Centocor and disclosed in U.S. patent application Ser. No. 10/280,716, hereby incorporated by reference into the present application, was used at several concentrations in the assay. F 105, human anti-gp 120 IgGl, also developed at Centocor, was used as a negative control antibody. Anti-SAA monoclonal antibody (clone REU-86.2, Research Diagnostics, Inc.) was used for SAA immunostaining.

A498 cells were cultured for 24 hrs in 96-well flat bottom tissue culture plates at a concentration of 6×104 cells/100 ul in DMEM complete media at 37oC./5% CO2. Cells were washed with PBS then starved for 1 hr in 100 ul of serum free DMEM media at room temp. A combination of IL-6 (100 ng/ml), sIL-6R (200 ng /ml) and IL-1b (1 ng/ml) mixture was incubated with serial dilutions of CNTO 328 or F105 control antibody for 30 minutes at room temperature. 100 ul of the mixture was added to the cells and incubated at 37oC./5% CO2 overnight.

### Detection of SAA by ELISA

The cell culture supernatant was assayed immediately for hSAA production using Biosource human SAA kit (Camrillo, Calif.) following manufacturer's instructions.

Results:

A498 cell line was induced to produce SAA

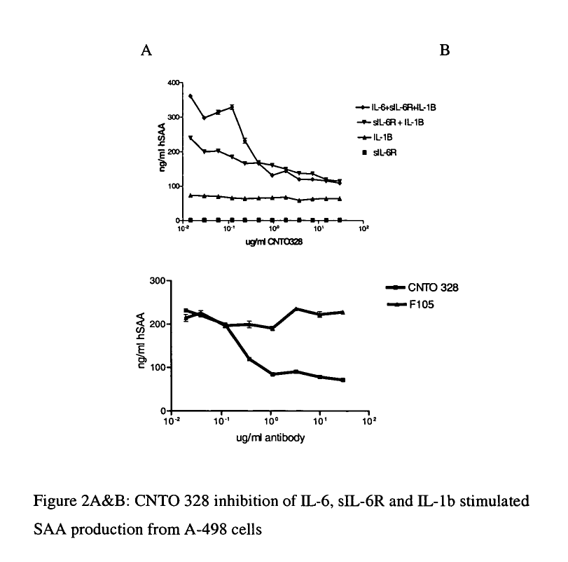

We tested whether renal tumor cells could be stimulated to produce SAA. Human renal tumor A498 cells produced significant levels of SAA (250-350 ng/ml) in culture when stimulated with IL-6, sIL-6R and IL-1β; this combination significantly induced SAA when compared to cells stimulated with IL-6 (<9 ng ml), sIL-6R (<9 ng/ml), IL-1b (<100 ng/ml), IL-1b+sIL−6R (200 ng/ml) (FIG. 2A).

CNTO 328 inhibited IL-6 induced SAA production from A498 cell line.

CNTO 328 completely inhibited IL-6/sIL-6R/IL-1β induced SAA production in a dose dependant fashion (FIG. 2A & B), suggesting that tumor cell-secreted SAA is a direct indicator of IL-6 activity, and in cancer patients CNTO 328 bioactivity can be monitored by reduction of SAA

1. A method of monitoring the response to anti-cytokine therapy in a patient undergoing such therapy by using Serum Amyloid A (SAA) protein as a biomarker, which comprises:
(a) determining the level of SAA protein in the serum or diseased tissue of a patient;
(b) administering the anti-cytokine therapy to the patient;
(c) measuring the level of SAA protein in the serum or diseased tissue of the patient and determining whether the anti-cytokine therapy is effective in reducing the level of SAA protein.

2. The method of claim 1 wherein the therapy is for the treatment of cancer and the SAA is employed as a tumor-responsive biomarker.

3. The method of claim 1 wherein the anti-cytokine therapy is selected from agents that neutralize or inhibit IL-6, TNFα, IL-1, IL-2, IFNγ, anti-ciliary neurotrophic factor, IL-I1, LIF, oncostatin M, or cardiotropin.

4. The method of claim 3 wherein the anti-cytokine therapy is anti-IL-6 therapy.

5. The method of claim 4 wherein the anti-IL-6 therapy is the administration of an anti-IL-6 neutralizing antibody.
